(19) United States
(12) Patent Application Publication
(10) Pub. No.: US 2023/0263912 A1
(43) Pub. Date: Aug. 24, 2023

(54) POLYSACCHARIDE COMPRISING A CHELATING GROUP SOLUBLE AT PHYSIOLOGICALPH AND USE THEREOF

(71) Applicants: MEXBRAIN, Villeurbanne (FR); CENTRE NATIONAL DE LA RECHERCHE SCIENTIFIQUE - CNRS, Paris (FR); UNIVERSITE CLAUDE BERNARD LYON 1, VILLEURBANNE CEDEX (FR)

(72) Inventors: Olivier TILLEMENT, FONTAINES SAINT-MARTIN (FR); François LUX, LYON (FR); Fabien ROSSETTI, VILLEURBANNE (FR); Marco NATUZZI, VILLEURBANNE (FR); Thomas BRICHART, VILLEURBANNE (FR); Laurent DAVID, LYON (FR); Paula NUNES DE OLIVEIRA, VILLEURBANNE (FR); Alexandra MONTEBAULT, RIVE-DE-GIER (FR)

(21) Appl. No.: 18/007,254

(22) PCT Filed: Jul. 29, 2021
(86) PCT No.: PCT/FR2021/051419

§ 371 Date: Jan. 27, 2023

(30) Foreign Application Priority Data
Jul. 29, 2020 (FR) 20 07997

(51) Int. Cl. A61K 49/12; C08B 37/08

(52) U.S. Cl. CPC A61K 49/128; C08B 37/003

(57) ABSTRACT

The invention relates to a statistical polysaccharide with a weight-average molecular weight of between 100 kDa and 1000 kDa of the following formula (I): in which: each Rc independently represents a group containing a chelating agent, each Z independently represents a binder which may be a single bond or a hydrocarbon chain containing between 1 and 12 carbon atoms, said chain able to be either linear or branched and being able to contain one or more unsaturations and being able to contain one or more heteroatoms, preferably chosen from nitrogen, oxygen, sulfur and atoms of the halogen family, x is between 0.005 and 0.7, preferably between 0.05 and 0.7, and preferentially between 0.2 and 0.6, y is between 0.01 and 0.7, preferably between 0.05 and 0.2, the ratio of y/x being greater than or equal to 0.05, preferably greater than or equal to 0.15, and the sum of x+y being greater than or equal to 0.30, preferably greater than or equal to 0.35. The invention also relates to the use of said polysaccharide in a dialysis process in order to capture at least one metal, in an MRI imaging process, in a brachytherapy process or in a process for marking foodstuffs to prevent forgeries.

### TECHNICAL FIELD

The present disclosure falls within the field of compounds bearing a chelating group. In particular, the invention relates to polysaccharides which are chelated or which can chelate one or more metals and to the uses thereof in various techniques such as dialysis for homeostasis, MRI imaging, brachytherapy or else the marking of foodstuffs to prevent forgeries.

### PRIOR ART

Polysaccharides are polymers derived from plant, fungal, animal or bacterial biomass. These polymers have very varied physiochemical properties and can be formed for a wide range of biological applications. Chemical modification of polysaccharides makes it possible to adjust their properties, in particular their solubility in an aqueous medium at pH values close to neutrality. Their functionality using highly specific chelating agents also enables applications in the biomedical field. Indeed, after grafting of these entities onto the structure of polysaccharides, the polymer could be used as a detoxifying agent for decontaminating organisms with respect to pathogenic metals, such as the context of the maintenance of homeostasis.

The maintenance of the homeostasis of the inside environment of the organism, that is to say of all the biological liquids and fluids of the organism, is necessary in order for said organism to function correctly. In many pathological conditions, systemic or local dysregulations of the homeostasis of metals and/or peptides or proteins have been demonstrated.

With regard to metals, chelation therapies, aimed at reducing the concentration of metal ions, have already been used for many years in the case of acute poisoning. Thus, a certain number of chelating agents are already accepted in human beings, each one being associated with a group of particular metals (G. Crisponi et al., Coordination Chemistry Reviews, 2015).

An increasing number of scientific studies bring to the fore the important role that metals might have in many neurological ailments, in particular iron, but also copper, zinc, manganese and even aluminum and lead (E. J. McAllum et al., J. Mol. Neurosci., 2016). This is in particular the case for neurodegeneration with iron overload, which is a rare disease associated with a genetic anomaly linked to iron accumulation in certain areas of the brain and for which, at the current time, there are only palliative treatments (S. Wiethoff et al., Handb. Clin. Neurol., 2017). Moreover, numerous studies have shown that iron has a tendency to accumulate in the brain with age (J. Acosta-Cabronero et al., Journal of Neuroscience, 2016). Wilson's disease is also a genetic disease which causes an accumulation of copper in the organism and leads to various problems, in particular hepatic and/or neurological problems (Anna Członkowskal et al., Nature Rev., 2018).

Several neurological diseases such as Alzheimer's disease, Parkinson's disease and Huntington's disease are also accompanied by an increase in the amount of iron in specific areas, causing cell damage and also oxidative stress (A. A. Belaidi et al., Journal of Neurochemistry, 2016). By way of example, Huntington's disease is a neurodegenerative disease which results in problems with movement, a cognitive decline and psychiatric problems. In this pathological condition, numerous oxidative stress markers are observed in the brain, which can be linked to a dysregulation of iron homeostasis (S. J. A. van den Bogaard et al., International Review of Neurobiology, 2013). The increase in iron level in several regions of the brain (putamen, caudate nucleus and pallidum) has thus been validated by several MRI studies, including that of Bartzorkis (G. Bartzorkis et al., Archives of Neurology, 1999).

In Alzheimer's disease in particular, interactions have been demonstrated between certain metal ions, in particular ions from metals such as zinc, iron or copper, and Aß peptides which can result in increased protein aggregation (Tougu et al. Metallomics, 2010).

It has thus been accepted that, in numerous proteinopathies, metal cations play an important role in the formation of abnormal configurations of certain proteins; in particular, some promote the formation of aggregates, fibrils or other solid deposits. In proteinopathies, there thus appears to be, locally, a double dysregulation of homoeostasis, namely dysregulation of the homeostasis of certain metals and dysregulation of the homeostasis of target molecules of protein type, responsible for aggregations and other solid deposits.

Patent application WO 2019/122790 discloses a medical device which can be introduced into the organism for maintaining the metal homeostasis for therapy purposes, said device comprising a chelating agent for extracting metals.

### Technical Problem

While medical devices that are satisfactory thus exist, it is still beneficial to improve the end results obtained to date. Moreover, a device comprising a chelating agent which can be used for capturing a metal in an organism can be potentially introduced into the organism in an already-chelated form. In the latter configuration, other very varied applications can be envisaged, such as magnetic resonance imaging (MRI), brachytherapy, or else the marking of foodstuffs to prevent forgeries.

Thus, an objective of the invention is to provide a polysaccharide comprising a chelating agent which can be introduced into an organism in a chelated or non-chelated form, for use thereof in various applications.

### SUMMARY OF THE INVENTION

The invention relates to a statistical polysaccharide with a weight-average molecular weight of between 100 kDa and 1000 kDa of formula I:

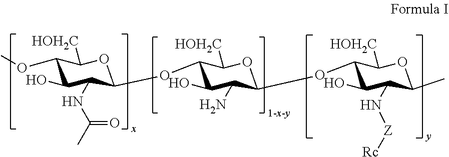

wherein:
each Rc independently represents a group comprising a chelating agent,
each Z independently represents a linker which can be a single bond or a hydrocarbon-based chain comprising between 1 and 12 carbon atoms, it being possible for said chain to be linear or branched and to comprise one or more unsaturations and to comprise one or more heteroatoms, preferably chosen from nitrogen, oxygen, sulfur and atoms of the halogen family,
x is between 0.005 and 0.7, preferably between 0.05 and 0.7, and preferentially between 0.2 and 0.6,
y is between 0.01 and 0.7, preferably between 0.05 and 0.2,
the y/x ratio being greater than or equal to 0.05, preferably greater than or equal to 0.15, and
the sum x+y being greater than or equal to 0.30, preferably greater than or equal to 0.35.

The invention also relates to the use of said polysaccharide in a dialysis process for capturing at least one metal.

The invention also relates to the use of said polysaccharide in an MRI imaging process.

The invention is also directed toward the use of said polysaccharide in a brachytherapy process.

The invention also relates to the use of said polysaccharide in a process for marking foodstuffs to prevent forgeries.

### BRIEF DESCRIPTION OF DRAWINGS

Other characteristics, details and advantages will emerge on reading the detailed description hereinafter, and on analyzing the appended drawings, wherein:

FIG. 1 is an HPLC-UV chromatogram obtained using a Phenomenex GFC-P 4000 SEC column, of a polysaccharide according to the invention.

FIG. 2 is a ¹H NMR spectrum of a polysaccharide according to the invention.

FIG. 3 is a graph representing the luminescence intensity at 594 nm as a function of the amount of europium added to a solution comprising a polysaccharide according to the invention.

FIG. 4 represents a graph of thermogravimetric analysis of a chitosan with a weight-average molecular weight of 600 kDa with a degree of acetylation of 0.5% and of a polysaccharide according to the invention grafted with DOTAGA (graph A) and a graph representing the derivative curves (graph B).

FIG. 5 represents the deconvolution of two Fourier transform infrared spectra.

FIG. 6 represents two graphs of the change in the amount of metals as a function of time in a solution dialyzed with ultrapure water (graph A) and a dialysis fluid comprising a polysaccharide according to the invention grafted with DOTAGA (graph B).

FIG. 7 represents three graphs of the change in the amount of metals as a function of time in a dialysis fluid comprising a polysaccharide according to the invention grafted with DOTAGA.

FIG. 8 is an HPLC-UV chromatogram obtained using a Phenomenex GFC-P 4000 SEC column, of a polysaccharide according to the invention (example 8).

FIG. 9 is a graph representing the absorbance at 425 nm as a function of the amount of iron added in a solution comprising a polysaccharide according to the invention (example 8).

### DETAILED DESCRIPTION OF THE INVENTION

As previously indicated, the invention relates to a statistical polysaccharide with a weight-average molecular weight of between 100 kDa and 1000 kDa of formula I:

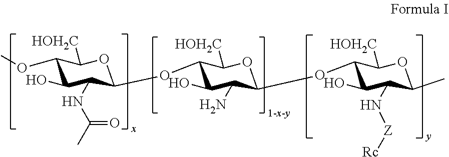

wherein:
each Rc independently represents a group comprising a chelating agent,
each Z independently represents a linker which can be a single bond or a hydrocarbon-based chain comprising between 1 and 12 carbon atoms, it being possible for said chain to be linear or branched and to comprise one or more unsaturations and to comprise one or more heteroatoms, preferably chosen from nitrogen, oxygen, sulfur and atoms of the halogen family,
x is between 0.005 and 0.7, preferably between 0.05 and 0.7, preferentially between 0.2 and 0.6, and more preferentially between 0.25 and 0.4,
y is between 0.01 and 0.7, preferably between 0.05 and 0.2,
the y/x ratio being greater than or equal to 0.05, preferably greater than or equal to 0.15, and
the sum x+y being greater than or equal to 0.30, preferably greater than or equal to 0.35.

It is understood that, in formula I above, several Rc groups may be present in the polysaccharide. These Rc groups may be identical to or different than one another. They are all independently chosen from groups bearing a chelating agent. The same is true for the linkers Z: several linkers Z may be present, and they may be identical to or different than one another.

According to one embodiment, in formula I, x is between 0.005 and 0.6, y is between 0.1 and 0.9,

the y/x ratio being greater than 0.16, and
the sum x +y being greater than 0.4.

Preferably, the polysaccharide according to the invention has a complexation constant of at least 10¹⁵ for a transition element d or f.

According to one embodiment, the polysaccharide of formula I is a polysaccharide of formula II:

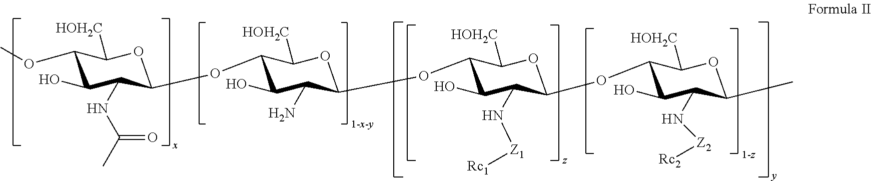

wherein:
Rc₁ and Rc₂ are different, and are groups comprising a chelating agent,
Z₁ and Z₂, which may be identical or different, are linkers which may be a single bond or a hydrocarbon-based chain comprising between 1 and 12 carbon atoms, it being possible for said chain to be linear or branched and to comprise one or more unsaturations and to comprise one or more heteroatoms, preferably chosen from nitrogen, oxygen, sulfur and atoms of the halogen family.
x is between 0.005 and 0.7, preferably between 0.05 and 0.7, preferentially between 0.2 and 0.6, and more preferentially between 0.25 and 0.4,
y is between 0.01 and 0.7, preferably between 0.05 and 0.2,
the y/x ratio being greater than or equal to 0.05, preferably greater than or equal to 0.15,
the sum x+y being greater than or equal to 0.30, preferably greater than or equal to 0.35, and
z is between 0.5 and 1.

In this specific embodiment, the polysaccharide of formula II may comprise:
a single type of group comprising a chelating agent, Rc₁, when z=1, or
2 types of groups comprising a chelating agent, Rc₁ and Rc₂, when 0.5≤z<1.

According to one embodiment, z is between 0.8 and 0.99 ; the Rc₁ group is therefore largely predominant.

According to another embodiment, in formula II, x is between 0.005 and 0.6,

y is between 0.1 and 0.9,
the y/x ratio being greater than 0.16,
the sum x+y being greater than 0.4, and
z is between 0.5 and 1.
The Groups of Rc Type (Rc, Rc₁ and Rc₂)

The term “group of Rc type” is intended to mean the Rc groups in the polysaccharide of formula I, and the Rc₁ and Rc₂ groups, when the R2 group is present, in the polysaccharide of formula II.

In accordance with the invention, the Rc, Rc₁ and Rc₂ groups are chelating agents. In other words, the Rc, Rc₁ and Rc₂ groups make it possible to chelate one or more metals while forming a complex.

Each of the Rc, Rc₁ and Rc₂ groups can contain one or more coordination sites. Preferably, the coordination site is a nitrogen or oxygen atom. Advantageously, each of the Rc, Rc₁ and Rc₂ groups comprises between 4 and 8 coordination sites, more advantageously between 6 and 8 coordination sites and, even more advantageously, each of the Rc, Rc₁ and Rc₂ groups comprises 8 coordination sites.

The term “coordination site” is intended to mean a single function capable of chelating a metal. For example, an amine function represents a site of coordination via the formation of a dative bond between the nitrogen atom and the metal, and a hydroxamic acid function also represents a site of coordination via the formation of a dative bond between the oxygen of the carbonyl entity and via a covalent bond with the oxygen of the N-oxide entity, the coordination site thus forming a five-membered ring.

In one embodiment of the invention, for the polysaccharide of formula I, each Rc group is independently chosen from the group consisting of DOTA (1,4,7,10-tetraazacyclododecane-N, N′, N″, N′″-tetraacetic acid), NOTA (1,4,7-triazacyclononane-1,4,7-triacetic acid), NODAGA (1,4,7-triazacyclononane-1-glutaric acid-4,7 diacetic acid), DOTAGA (2-(4,7,10-tris(carboxymethyl)-1,4,7,10-tetraazacyclododecan-1-yl)pentanedioic acid), DOTAM (1,4,7,10-tetra-kis(carbamoylmethyl)-1,4,7,10 tetraazacyclododecane), NOTAM (1,4,7-tetra-kis(carbamoylmethyl)-1,4,7-triazacyclononane), DOTP (1,4,7,10-tetra-azacyclododecane 1,4,7,10-tetrakis(methylene phosphonate), NOTP (1,4,7-tetrakis(methylene phosphonate)-1,4,7-triazacyclononane), TETA (1,4,8,11-tetra-azacyclotetradecane-N,N′,N″,′″-tetraacetic acid), TETAM (1,4,8,11-tetra-azacyclotetradecane-N,N′,N″,N′″-tetrakis(carbamoylmethyl), DTPA (diethylene 20 triaminopentaacetic acid) and DFO (deferoxamine), and preferably from the group consisting of DOTAGA, DFO, DOTAM and DTPA and more preferably the Rc group is DOTAGA.

In one embodiment of the invention, for the polysaccharide of formula II, Rc₁ and Rc₂ are independently chosen from the group consisting of DOTA, NOTA, NODAGA, DOTAGA, DOTAM, NOTAM, DOTP, NOTP, TETA, TETAM, DTPA and DFO, preferably from the group consisting of DOTAGA, DFO, DOTAM and DTPA.

According to one embodiment, for the polysaccharide of formula II, the Rc₁ group is DOTAGA, and preferably z=1.

According to one embodiment, for the polysaccharide of formula II, the Rc₁ group is DOTAGA and the Rc₂ group is DFO.

The linkers of Z type (Z, Z₁ and Z₂)

The term “linker of Z type” is intended to mean the linkers Z in the polysaccharide of formula I, and the linkers Z₁ and Z₂, when the linker Z₂ is present, in the polysaccharide of formula II.

The choice of the linkers Z, Z₁ and Z₂ in formulae I and II depends essentially on the Rc, Rc₁ and Rc₂ groups and on the metal to be chelated. This is because, for steric reasons in particular, the Rc, Rc₁ and Rc₂ groups may be more or less close to the 6-membered ring of the nitrogen of the glucosamine unit.

Preferably, in formula I, each Z is independently a single bond or a hydrocarbon-based chain comprising between 1 and 12 carbon atoms, it being possible for said chain to be linear or branched and to comprise one or more unsaturations and to comprise one or more heteroatoms, preferably chosen from nitrogen, oxygen, sulfur and atoms of the halogen family.

According to one embodiment, in formula I, each Z is independently selected from the group consisting of: a bond, a linear or branched alkyl chain comprising between 1 and 12 carbon atoms, and a linear or branched alkenyl chain comprising between 2 and 12 carbon atoms,

it being possible for said alkyl and alkenyl chains to be interrupted with one or more C₆-C₁₀ aryl groups, and/or with one or more heteroatoms or groups selected from the group consisting of —O—, —S—, —C(O)—, —NR′—, —C(O)NR′13 , —NR′—C(O)—, —NR′—C(O)—NR′—, —NR′—C(O)—O—, —O—C(O)NR′, —C(S)NR′—, —NR′—C(S)—, —NR′—C(S)—NR′, it being possible for said alkyl and alkenyl chains to be substituted with one or more groups selected from the group consisting of halogens, —OR′, —COOR′, —SR′, —NR′₂, each R′ is independently H or a C₁-C₆ alkyl.

Advantageously, in formula I, each Z is independently selected from the group consisting of: a bond and a linear or branched alkyl chain comprising between 1 and 12 carbon atoms,

it being possible for said alkyl chain to be interrupted with one or more C₆-C₁₀ aryl groups, and/or with one or more heteroatoms or groups selected from the group consisting of —O—, —S—, —C(O)—, —NR′—, —C(O)NR′—, —NR′—C(O)—, —C(S)NR′—, —NR′—C(S)—, —NR′—C(S)—NR′,
each R′ is independently H or a C₁-C₆ alkyl.

In one particular embodiment, each Z is an alkyl chain comprising between 1 and 12 carbon atoms.

In another particular embodiment, each Z is a polyethylene glycol (PEG).

Preferably, in formula II, Z₁ and Z₂ are independently a single bond or a hydrocarbon-based chain comprising between 1 and 12 carbon atoms, it being possible for said chain to be linear or branched and to comprise one or more unsaturations and to comprise one or more heteroatoms, preferably chosen from nitrogen, oxygen, sulfur and atoms of the halogen family.

According to one embodiment, in formula II, Z₁ and Z₂ are independently selected from the group consisting of: a bond, a linear or branched alkyl chain comprising between 1 and 12 carbon atoms, and a linear or branched alkenyl chain comprising between 2 and 12 carbon atoms,

it being possible for said alkyl and alkenyl chains to be interrupted with one or more C₆-C₁₀ aryl groups, and/or with one or more heteroatoms or groups selected from the group consisting of —O—, —S—, —C(O)—, —NR′—, —C(O)NR′—, —NR′—C(O)—, —NR′—C(O)—NR′—, —NR′—C(O)—O—, —O—C(O)NR′, —C(S)NR′—, —NR′—C(S)—, —NR′—C(S)—NR′, it being possible for said alkyl and alkenyl chains to be substituted with one or more groups selected from the group consisting of halogens, —OR′, —COOR′, —SR′ and —NR′₂,
each R′ is independently H or a C₁-C₆ alkyl.

Advantageously, in formula II, Z₁ and Z₂ are independently selected from the group consisting of: a bond and a linear or branched alkyl chain comprising between 1 and 12 carbon atoms,

it being possible for said alkyl chain to be interrupted with one or more C₆-C₁₀ aryl groups, and/or with one or more heteroatoms or groups chosen from the group consisting of —O—, —S—, —C(O)—, —NR′—, —C(O)NR′—, —NR′—C(O)—, —C(S)NR′—, —NR′—C(S)—, —NR′—C(S)—NR′,
each R′ is independently H or a C₁-C₆ alkyl.

In one particular embodiment, Z₁ and/or Z₂ is an alkyl chain comprising between 1 and 12 carbon atoms.

In another particular embodiment, Z₁ and/or Z₂ is a polyethylene glycol (PEG).

### Monomeric Units of the Polysaccharide According to the Invention

The polysaccharide according to the invention is composed of three monomeric units, namely a unit A of N-acetylglucosamine type, a unit B of glucosamine type and a unit of type C of glucosamine type functionalized with a chelating agent (of Rc type) linked by a linker (of Z type) to the nitrogen of the glucosamine.

The polysaccharide according to the invention is a random polymer. In other words, the linking of the various monomeric units A, B and of type C is random.

In formulae I and II, x represents the proportion of units A and x can be between 0.05 and 0.7; preferably, x is between 0.2 and 0.6; more preferably, x is between 0.25 and 0.4.

In formulae I and II, y represents the proportion of units of type C and y can be between 0.01 and 0.7.

The remainder of the monomeric units of formulae I and II are units B. Thus, in formulae I and II, the proportion of units B is equal to 1-x-y.

In accordance with the invention, in formulae I and II, the y/x ratio can be greater than or equal to 0.05, preferably greater than or equal to 0.15. Indeed, the effectiveness of the product is determined by the number of chelation sites, directly linked to the number of metals required for example for detection by imaging.

In order to be able to be introduced in liquid form into an organism while at the same time producing the desired effects, the polysaccharide according to the invention must be soluble at physiological pH. To do this, the sum x+y can be greater than or equal to 0.30, preferably greater than or equal to 0.35.

The combination of the particular ratio between the number of units A and 25 the number of units of type C and of the sum of the proportion of units A and of the proportion of units of type C makes it possible to attain a chelation and a solubility which are suitable for allowing the use of the polysaccharide according to the invention in varied fields, such as dialysis for capturing at least one metal, MRI imaging, brachytherapy and the marking of foodstuffs to prevent forgeries.

Advantageously, x is between 0.2 and 0.6; more preferably, x is between 0.25 and 0.4.

Advantageously, y is between 0.01 and 0.7, preferably between 0.05 and 0.2.

In accordance with the invention, z is between 0.5 and 1. In other words, the units of type C may be exclusively units comprising Z₁ as linker and Rc₁ as group bearing a chelating agent.

The polysaccharide according to the invention has a weight-average molecular weight of between 100 kDa and 1000 kDa ; advantageously, the weight-average molecular weight of the polysaccharide according to the invention is between 250 kDa and 750 kDa, more advantageously between 400 kDa and 600 kDa and even more advantageously between 450 kDa and 550 kDa.

According to one embodiment, the polysaccharide is chosen from the following polysaccharides:
polysaccharide of formula II where z=1, Rc₁ is DOTAGA and Z₁ is a bond;
polysaccharide of formula II where z=1, Rc₁ is DTPA and Z₁ is a bond; and
polysaccharide of formula II where 0.5 z≤1, Rc₁ is DOTAGA and Z₁ is a bond, and Rc₂ is DFO and Z₂ is selected from the group consisting of: a bond and a linear or branched alkyl chain comprising between 1 and 12 carbon atoms, it being possible for said alkyl chain to be interrupted with one or more C₆-C₁₀ aryl groups, and/or with one or more heteroatoms or groups selected from the group consisting of —O—, —S—, —C(O)—, —NR′—, —C(O)NR′—, —NR′—C(O)—, —C(S)NR′—, —NR′—C(S)—, —NR′—C(S)—NR′,
   each R′ is independently H or a C₁-C₆ alkyl.

Process for obtaining the polysaccharide according to the invention

The polysaccharide of formulae I or II according to the invention can be obtained from chitosan, which is optionally partially acetylated, in particular by acetylation of a portion of the amine functions and then functionalization of at least one portion of the amine functions still present after the acetylation.

In one particular embodiment, the process for obtaining the polysaccharide according to the invention comprises at least the following successive three steps:
Step 1: solubilization of a chitosan in an acid solution at a pH of between 4 and 5;
Step 2: partial acetylation of the amine functions of the chitosan solubilized in step 1 (formation of the units A);
Step 3: functionalization of at least one portion of the amine functions still present at the end of step 2 (formation of the units of type C).

When the polysaccharide comprises several different groups of Rc type, step 3 of the process can be repeated several times. For example, if the polysaccharide of formula II comprises an Rc₁ group and an Rc₂ group, the process can comprise two steps 3 of functionalization of at least one portion of the amine functions, a first for introducing the Rc₁ groups and a second for introducing the Rc₂ groups.

Step 3 can be subdivided into several substeps, in particular when the linker Z is a hydrocarbon-based chain as defined above.

In the embodiments where the linker of Z type is a hydrocarbon-based chain as defined above, step 3 can comprise a substep 3-1 consisting in grafting said hydrocarbon-based chain onto at least one portion of the amine functions still present at the end of step 2, then a substep 3-2 consisting in grafting the group of Rc type onto said hydrocarbon-based chain. Alternatively, step 3 does not comprise a substep. In this alternative, said hydrocarbon-based chain is coupled with the 20 group of Rc type prior to step 3, and said step 3 is then carried out with a molecule comprising the group of Rc type and said hydrocarbon-based chain.

Alternatively, the polysaccharide according to the invention can be obtained from a chitosan having the desired degree of acetylation; thus, in this embodiment, the units A are already present and do not need to be formed. In this embodiment, the process for obtaining the polysaccharide according to the invention comprises at least the following two successive steps:
Step 1b: solubilization of a partially acetylated chitosan (unit A) in an acid solution at a pH of between 4 and 5;
Step 2b: functionalization of at least one portion of the amine functions of said partially acetylated chitosan solubilized in step 1b (formation of the units of type C).

In the same way as above, step 2b of the process can be repeated several times, when several different groups of Rc type are present.

In the same way as above for said step 3, said step 2b can be subdivided into several substeps, in particular when the linker of Z type is a hydrocarbon-based chain as defined above.

In the embodiments where the linker of Z type is a hydrocarbon-based chain as defined above, step 2b can comprise a substep 2b-1 consisting in grafting said hydrocarbon-based chain onto at least one portion of the amine functions, then a substep 2b-2 consisting in grafting the group of Rc type onto said hydrocarbon-based chain. Alternatively, step 2b does not comprise a substep. In this alternative, said hydrocarbon-based chain is coupled with the group of Rc type prior to step 2b, and said step 2b is then carried out with a molecule comprising the group of Rc type and said hydrocarbon-based chain.

When the polysaccharide according to the invention is in chelated form, the process can comprise a step 4 of chelation of the polysaccharide with at least one metal.

Use of the Polysaccharide According to the Invention

The polysaccharide according to the invention can be used in various applications by virtue of its solubility at physiological pH. The groups of Rc type make it possible to chelate a metal; consequently, it can be envisaged to introduce said polysaccharide into an organism in two different forms, (i) in chelated form, that is to say that the polysaccharide chelates at least one metal, or (ii) in free form, that is to say that the polysaccharide does not chelate metal. When the polysaccharide is in free form, this means that less than 5% of the chelating groups of Rc type are complexed; in particular, less than 5% of the chelating groups of Rc type chelate ions of interest. When the polysaccharide is in chelated form, this means that at least 80% of the chelating groups of Rc type are complexed with the metal(s) of interest.

As indicated above, the invention also relates to the use of said polysaccharide in a dialysis process for capturing at least one metal, in an MRI imaging process, in a brachytherapy process or else in a process for marking foodstuffs to prevent forgeries.

When the polysaccharide is used in a dialysis process for capturing at least one metal, said polysaccharide is in free form, to allow the capturing of at least one metal.

When the polysaccharide is used in an MRI imaging process, in a brachytherapy process or else in a process for marking foodstuffs to prevent forgeries, said polysaccharide chelates at least one metal.

The polysaccharide according to the invention can be used as such, or can be formulated in a composition. In the case where the polysaccharide is formulated in a composition, said polysaccharide is advantageously in hydrogel form.

Use in a Dialysis Process for Capturing at Least One Metal

In one particular embodiment, the dialysis is a MARS dialysis or a dialysis of CSF.

When the polysaccharide is used in a dialysis process for capturing at least one metal, the metal preferably belongs to the group consisting of copper, iron, lead, zinc, aluminum, gadolinium and manganese, and more preferentially is in the group consisting of copper, iron and lead.

When the polysaccharide is used in a dialysis process for capturing at least one metal, the groups of Rc type are preferably chosen from the group consisting of DOTAGA, DFO, DOTAM and DTPA.

When the polysaccharide is used in a dialysis process for capturing at least one metal, x is preferably between 0.25 and 0.4.

When the polysaccharide is used in a dialysis process for capturing at least one metal, y is preferably between 0.05 and 0.2.

When the polysaccharide is used in a dialysis process for capturing at least one metal, the y/x ratio is preferably between 0.15 and 1.5.

When the polysaccharide is used in a dialysis process for capturing at least one metal, the sum x +y is preferably between 0.35 and 0.8.

Use in an MRI Imaging Process

When the polysaccharide is used in an MRI imaging process, said polysaccharide chelates at least one metal. The metal preferably belongs to the group consisting of gadolinium, iron, manganese and dysprosium and more preferentially, the metal is gadolinium.

When the polysaccharide is used in an MRI imaging process, z is preferably equal to 1 and the Rc₁ group is preferably DOTAGA.

When the polysaccharide is used in an MRI imaging process, x is preferably between 0.05 and 0.35.

When the polysaccharide is used in an MRI imaging process, y is preferably between 0.1 and 0.7.

Use in a Brachytherapy Process

When the polysaccharide is used in a brachytherapy process, said polysaccharide chelates at least one metal. The metal is a radioactive isotope.

Thus, when the polysaccharide is used in a brachytherapy process, the metal preferably belongs to the group of radioactive isotopes consisting of ¹⁷⁷Lu, ¹⁶⁶ Ho, ²¹²Bi, ²¹³Bi, ⁹⁰Y and ²²⁵At.

When the polysaccharide is used in a brachytherapy process, z is preferably equal to 1 and the Rc₁ group is preferably chosen from the group consisting of DOTAGA.

Use in a Process for Marking Foodstuffs to Prevent Forgeries

When the polysaccharide is used in a process for marking foodstuffs to prevent forgeries, said polysaccharide chelates at least one metal. The metal preferably belongs to the group consisting of lanthanides and bismuth.

When the polysaccharide is used in a process for marking foodstuffs to prevent forgeries, the groups of Rc type are preferably chosen independently from the group consisting of DOTA, NOTA, NODAGA, DOTAGA, DOTAM, NOTAM, DOTP, NOTP TETA and TETAM. For the use of the polysaccharide according to the invention in a process for marking foodstuffs to prevent forgeries, the cyclic chelating agents are in fact advantageous in order to avoid transmetallization.

### EXAMPLES

Materials and Methods

Acetic anhydride (>99%) and 1,2-propanediol were supplied by Sigma-Aldrich (France), DOTAGA anhydride, DTPA-bisanhydride and p-NCS-Bz-DFO (N1-hydroxy-N1-(5-(4-(hydroxy(5-(3-(4-isothiocyanatophenyl)thio-ureido)pentyl)amino)-4-oxobutaneamido)pentyl)-N4-(5-(N-hydroxy-acetamido)pentyl)succinamide) were supplied by CheMatech (France), DMSO was supplied by Fischer Chemicals), and the ultrapure water (Milli-Q water) was obtained by virtue of an Eolia filtration system. Chitosan was supplied by Mahtani, GdCl₃ by Sanofi, KBr and all the metal salts used in example 4 (Al, Mn, Cu, Pb and Zn nitrates) by Sigma-Aldrich (France).

The tangential flow filtration is carried out by means of a Sartoflow Smart tangential flow filtration machine with Sartocon Slice 200 polyethersulfone membranes with a cutoff threshold of 100 kDa.

The HPLC-UV is carried out with an Agilent 1200 apparatus with a DAD detector. The size exclusion column used is a Polysep-GFC-P-4000 with a 0.1 M acetic acid/ammonium acetate buffer as eluent. The detection is performed using a UV detector at a wavelength of 295 nm. The substance to be analyzed is injected at a concentration of approximately 10 g.l⁻¹

The UV-visible spectrophotometry studies were carried out on a Cary 50 UV-VIS apparatus from the company Varian.

The proton NMR spectroscopy was carried out on a Brucker Avance III 400 MHz at ambient temperature. All the samples were dissolved at 8 mg/ml in deuterated water (D20) comprising 5 μL of 12 N HCl and placed in 5 mm NMR tubes. TMPSA is used as internal reference.

The elemental analysis was carried out at the Institut des Sciences

Analytiques [Institute of Analytical Sciences], UMR 5280, Pole Isotopes & Organique, 5 rue de la Doua 69100 Villeurbanne.

The time-resolved luminescence studies were carried out using equipment of Cary Eclipse type from Agilent.

The relaxivity at 1.5 T was carried out with equipment of Minispec mq-60 type from Brucker (Karlsruhe, Germany).

### Example 1

Preparation of a Polysaccharide According to the Invention with a Degree of Acetylation of 40% (x=0.4) and a Degree of DOTAGA Grafting of 10% (y =0.1) Starting from a Chitosan with an Average Weight of 250 kDa

In step 1, 60 g of chitosan, 4 l of ultrapure water and 45 ml of glacial acetic acid are introduced into a 10 l reactor and stirred for a period of 16 hours at a pH of 4.5±0.5. A pale yellow solution is obtained.

In step 2, 1.2 l of 1,2-propanediol are added to the pale yellow solution obtained in step 1 and stirring is maintained for 1 h. A solution composed of 14 ml of acetic anhydride dissolved in 600 ml of 1,2-propanediol is then added slowly over the course of 10 min in order to obtain uniform acetylation along the polymeric chain; the reaction medium is kept stirring for 4 h.

The degree of acetylation can be determined by elemental analysis. The nonacetylated unit of the polysaccharide (unit B) has a molar mass of 161.2 g.mol⁻¹ (C₆NO₄H₁₁), whereas the acetylated unit (unit A) has a molar mass of 203.2 g.mol⁻¹ (C₈NO₅H₁₃). The elemental analysis of the polysaccharide obtained at the end of the acetylation step 2 is the following: C 39.22%; H 7.55% and N 6.77%, which corresponds to a degree of acetylated units (units A) of 40% (x=0.4)

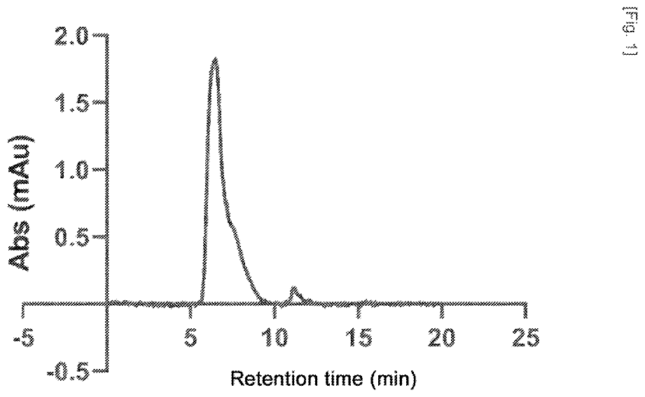

In step 3, 2 l of the solution obtained in the acetylation step 2 are placed in a reactor with stirring. 120 g of DOTAGA anhydride are then added and the stirring is maintained for 16 h. At the end of this reaction, the solution is diluted 10-fold in ultrapure water and purified by tangential flow filtration using a 100 kDa membrane. After a first step of reconcentrating down to 16 l, the solution is filtered using 480 l of a 0.1 M solution of acetic acid at constant volume (16 l) followed by 320 l of ultrapure water and by another step of reconcentrating down to 8 l. HPLC-UV makes it possible to verify that the DOTAGA has indeed been removed (FIG. 1). The peak at around 7 min corresponds to the polymer, whereas the peak at around 11 min corresponds to the non-grafted DOTAGA. The solution at a polysaccharide concentration of 10 g/l is then filtered through a nylon membrane (0.4 μm) before lyophilization.

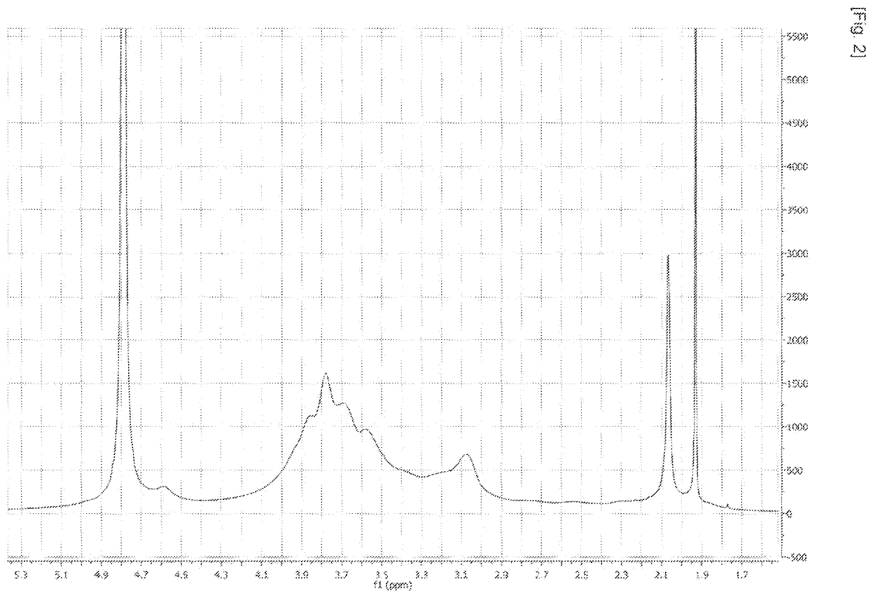

Proton NMR makes it possible to determine the degree y of functionalization with DOTAGA on the polysaccharide given knowledge of the degree x of acetylation. The non-grafted and non-acetylated unit (unit B) consists of 7 protons, covalently bonded to carbon atoms, having a chemical shift of between 2.9 and 4.3 ppm. The acetylated unit (unit A) has these same 7 protons and also 3 protons, covalently bonded to a carbon atom, which are present on the acetyl, and characterized by a chemical shift of between 2 and 2.2 ppm. Finally, the unit grafted with DOTAGA (unit C) includes 34 protons, covalently bonded to carbon atoms, 32 of which integrate between 2.9 and 4.3 ppm and two of which integrate between 2 and 2.2 ppm. The NMR spectrum represented in FIG. 2 makes it possible, by virtue of the integrations of the various peaks, to determine the values of y by virtue of the following equation:

$$\frac{{Area}_{2.9 - 4.3}}{{Area}_{2 - 2.2}} = \frac{7 + 25 y}{3 x + 2 y} \qquad [ Math . 1 ]$$

The degree of grafted unit (unit C) is approximately 0.1.

Thus, the polysaccharide obtained has a degree of unit A of approximately 0.4 (x=0.4), a degree of unit B of approximately 0.5 (1−x−y=0.5) and a degree of unit C of approximately 0.1 (y=0.1).

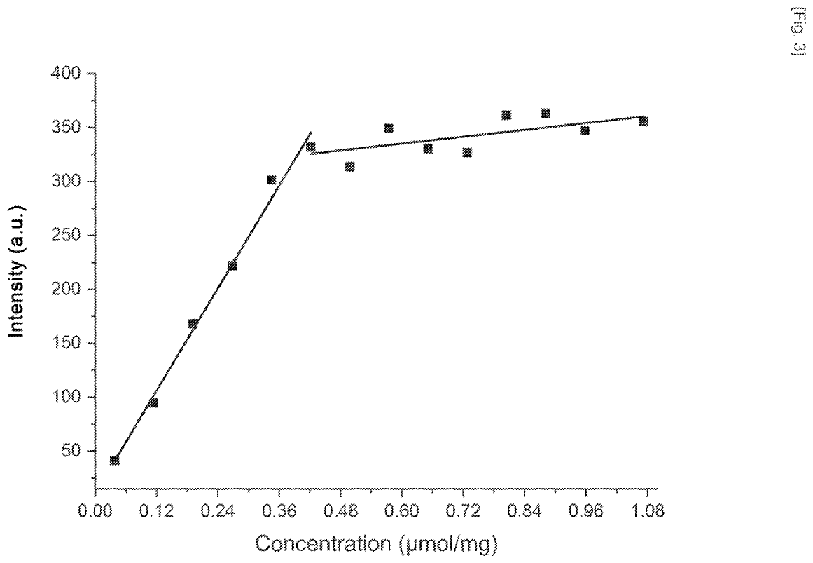

The degree of grafted units (unit C) can also be determined by fluorescence with europium. Europium in fact has a luminescence mainly centered around 590 (⁵D₀->⁷F₁) and 615 nm (5D₀->7F₂). This luminescence is extinguished when the europium ion is coordinated only with water molecules. The principle of the method for determining the degree of grafted units is that of adding increasing amounts of europium, in order for the latter to be chelated, the luminescence then increases, when all the chelation sites are filled, the luminescence reaches a plateau. In practice, the polysaccharide obtained at the end of step 3 was placed in an acetate buffer at pH 5, and a europium chloride salt dissolved in the acetate buffer was then added. A curve of quantitative analysis is then traced by exciting a 396 nm and noting the emission at 590 nm (FIG. 3). This quantitative analysis makes it possible to work back to an amount of chelate of 0.4 μmol per mg of polymer, i.e. a degree of approximately 10% (y=0.1).

### Example 2

Preparation of a Polysaccharide According to the Invention with a Degree of Acetylation of 0.5% (x=0.005) and a Degree of DOTAGA Grafting of 60% (y=0.6) Starting from a Chitosan with an Average Weight of 600 kDa having a Degree of Acetylation of 0.5%

In step 1 b, 3 g of a chitosan with an average weight of 600 kDa and having a degree of acetylation of 0.5%, 150 ml of ultrapure water and 2.1 ml of glacial acetic acid are introduced into a 1 l reactor and stirred for a period of 16 hours at a pH of 4.5±0.5. A pale yellow solution is obtained.

Following the solubilization step 1b, 150 ml of 1,2-propanediol are added to the preceding solution and the stirring is maintained for 1 h. 11 g of DOTAGA anhydride are then added and the stirring is maintained for 16 h. At the end of this reaction, the light brown solution is diluted 10-fold in ultrapure water and purified by tangential flow filtration using a 100 kDa membrane. This first purification cycle is followed by a second using the ultrapure water for a purification by a factor of 50.

The subsequent filtrations are carried out in 0.01 M HCl for a final purification factor of 1250.

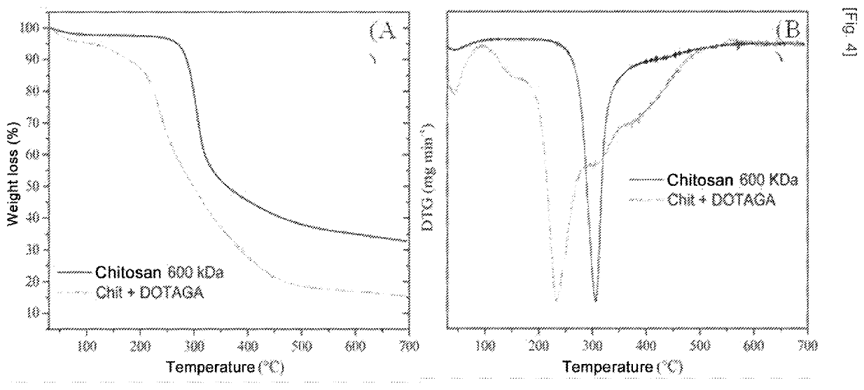

The grafting with DOTAGA (units C) were shown by thermogravimetric analysis using a TA Instruments TGA-Q500 apparatus with heating at 10° C./min, under inert atmosphere, from 30° C. to 700° C. The grafting of the DOTAGA induces a decrease in the degradation temperature proportional to the degree of grafting. Thus, comparison of the thermogravimetric analyses (graph (A) of FIG. 4) of the starting chitosan and of the polysaccharide according to the invention and of the derivative curves (graph (B) of FIG. 4) makes it possible to deduce that the functionalization step has indeed been effective.

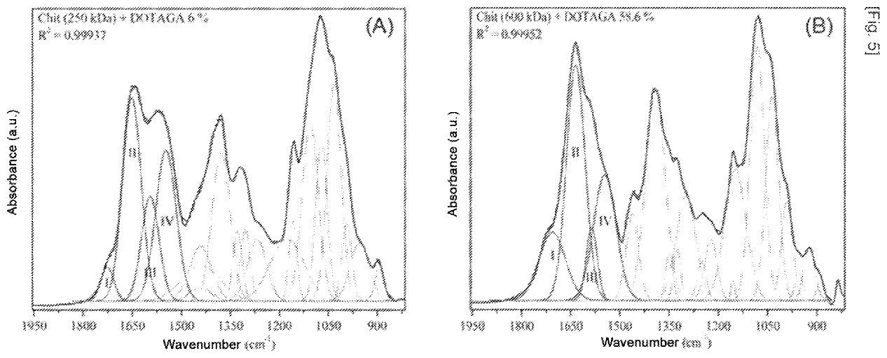

The IR spectra are normalized using the band having a maximum absorption (1072 cm⁻1) (FIG. 5). It is possible to validate the formation of amide bonds and to quantify them by deconvolution of the spectrum and by looking at the area of the band at 1590 cm⁻1 (amine NH₂ bond stretching band). The calculation is carried out by comparing the areas of the spectral bands located around 1590 cm¹. To determine the factor z=100×(1-x-y) of the following equation, it is necessary to determine the degree of functionalization of the chitosan/DOTAGA product by other techniques. In this example, the IR spectrum of chitosan+DOTAGA (6.0%) was used as reference spectrum (spectrum (A)).

$${D}_{S} = 100 - ( \frac{\frac{{A}_{( 1590 )}^{′}}{{A}_{T}^{′}}}{\frac{{A}_{ref ( 1590 )}}{{A}_{T ref}}} \times z )$$

Where Dₛ is the degree of substitution of the chitosan analyzed, A′₍₁₅₉₀₎ is the area of the band at 1590 cm⁻¹ of the IR spectrum of the chitosan to be analyzed, A′_{T} is the total area of the IR spectrum of the chitosan to be analyzed, A_{ref (1590)} is the area of band IV at 1590 cm⁻¹ of the IR spectrum of the reference chitosan, A_{Tref} is the total area of the reference chitosan spectrum and z is the percentage of free amine groups of the chitosan (x=0.02; y=0.06) used as reference (z=92). A degree of DOTAGA grafting close to 60% is then obtained (spectrum (B)).

### Example 3

Preparation of a Polysaccharide According to the Invention with a Degree of Acetylation of 60% (x=0.6) and a Degree of DTPA Grafting Close to 5% (y≈0.05) starting from a chitosan with an average weight of 250 kDa

In step 1, 0.25 g of chitosan, 25 ml of ultrapure water and 0.18 ml of glacial acetic acid are introduced into a 250 ml reactor and stirred for a period of 16 hours at a pH of 4.5±0.5. A pale yellow solution is obtained.

In step 2, 5 ml of 1,2-propanediol are added to the pale yellow solution obtained in step 1 and the stirring is maintained for 1 h. A solution composed of 0.045 ml of acetic anhydride dissolved in 5 ml of 1,2-propanediol is then added slowly over the course of 10 min in order to obtain uniform acetylation along the polymeric chain; the reaction medium is kept stirring for 4 h.

In step 3, a solution composed of 0.346 g of DTPA-bisanhydride dissolved in 15 ml of 1,2-propanediol and 16.58 μl of ultrapure water is kept stirring for 1.5 h. This solution is then added to the solution obtained in step 2 and the stirring is maintained for 16 h. At the end of this reaction, the solution is diluted 10-fold by adding ultrapure water, and purified by tangential flow filtration using a 100 kDa membrane. The following filtrations are carried out in ultrapure water for a final purification factor of 6250. The solution at a concentration of 5 g/l is then lyophilized.

### Example 4

Use of the Polymer of Example 1 for Capturing Metals

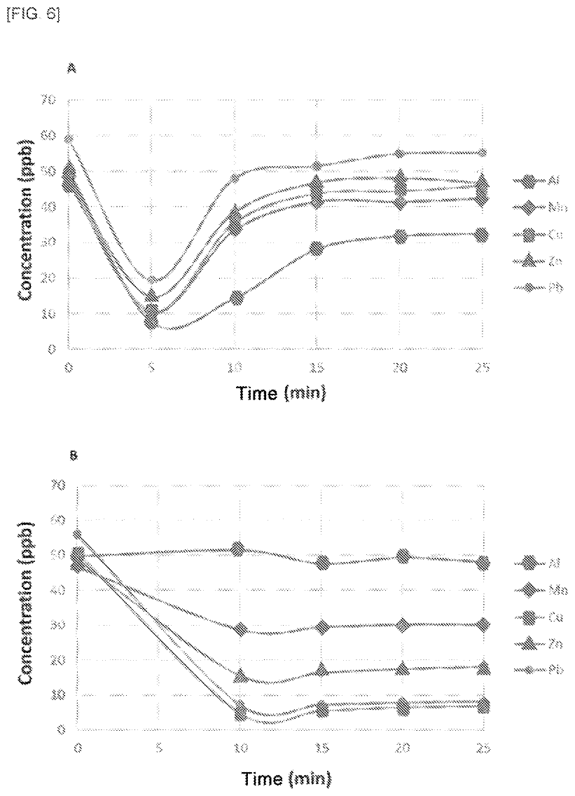

The polysaccharide synthesized in example 1 was formulated to be incorporated into a hemodialysis dialysate for the purpose of demonstrating its ability to perform a metal extraction in the context of conventional hemodialysis. For the requirements of the experiment, a Dialife DIAPHO6 high-flow hemodialyser, a Cole-Parmer Masterflex 7555-05 peristatic pump, and Masterflex L/S PharMed BPT Tubing, L/S #16 were used. The metal solution used here was prepared so as to have a content of approximately 50 ppb of Cu, of Zn, of Pb, of Al and of Mn in 5 l of water. The flow rate is 200 ml/min and the temperature is 37° C. A dialysate comprising only water was compared to a dialysate comprising 0.001 mol.l⁻¹ of DOTAGA, corresponding to 1.6 g/l of the polysaccharide synthesized in example 1. For all the metals, with the exception of aluminum, a greater capture is observed for the dialysis fluid comprising the polymer that is functionalized 25 min after the beginning of the dialysis by ICP/MS analysis (FIG. 6). The amount of metal present in the solution to be dialyzed is in fact 30 versus 42 ppb for manganese, 18 versus 46 ppb for zinc, 7 versus 46 ppb for copper and 8 versus 55 ppb for lead after dialysis with the solution of the polysaccharide of example 1 in comparison with a dialysis carried out with an aqueous solution not containing polysaccharide according to the invention.

### Example 5

Use of the Polymer of Example 1 in a Process for Dialyzing Pig Blood for Capturing Metals

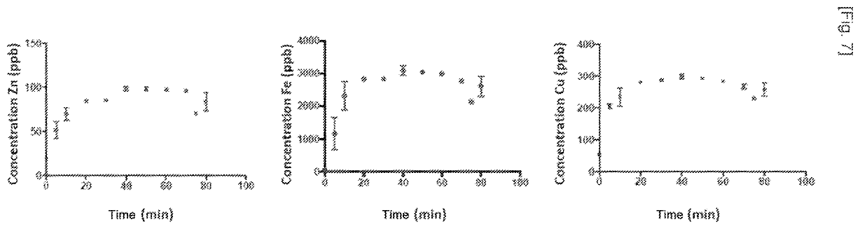

In order to demonstrate the capturing capacity in biological medium, an experiment similar to that described in example 4 was carried out with pig blood in the place of the aqueous metal solution presented in example 4. For the requirements of the experiment, a Dialife 20HP high-flow hemodialyser, three Cole Parmer Masterflex peristaltic pumps (7555-05, 7523-80 and 7523-90), Masterflex L/S PharMed BPT Tubing, L/S #16 and Ismatec SC0328 PharMed BPT Tubing were used. 10 l of pig blood were dialyzed at different flow rates: 200 ml/min (from 0 to 10 min and from 70 to 80 min), 100 ml/min (from 10 to 30 min) and 50 ml/min (from 30 to 70 min) respectively. The dialysis fluid was formulated with the polysaccharide synthesized in example 1 at a concentration of 0.001 mol.l⁻¹. The results of the study after ICP/MS analysis are presented in FIG. 7. A large increase in contents of Zn, Fe and Pb is observed in the dialysis fluid comprising the polysaccharide synthesized in example 1 in comparison with a dialysis carried out without the polysaccharide synthesized in example 1. The amount of metal present in the dialysate solution is thus 83 versus 20 ppb for zinc, 257 versus 55 ppb for copper and 2604 versus 62 ppb for iron.

### Example 6

Addition of Gadolinium for Obtaining an MRI-Imageable Polymer

After the purification of example 2, 4.9 g of gadolinium chloride hexahydrate are added to the solution, the pH is then adjusted to 5 and the solution is left to stir at 80° C. for 48 hours. The pH is continually adjusted to 5 with 0.5 M sodium hydroxide until it becomes stable, which is an indication that the DOTAGA has complexed all the Gd³⁺ ions.

### Example 7

Use of the Polysaccharide Complexed with Gadolinium

The polysaccharide according to the invention complexed with gadolinium in example 6 was used in an MRI imaging process.

The efficiency of the polymer complex with gadolinium for MRI was evaluated by measuring its longitudinal relaxivity (r¹=15.2 mM⁻¹.s⁻¹ by gadolinium) and its transverse relaxivity (r₂=21.5 mM⁻¹.s⁻¹ by gadolinium) at 37° C. in a field of 1.5T. The r₂/r₁ ratio is 1.4, which is typical of a positive contrast agent. The longitudinal relaxivity is a little less than four times greater than that of the commercial contrast agents (˜4 mM⁻¹.s⁻¹).

### Example 8

Preparation of a Polysaccharide According to the Invention with a Degree of Acetylation of 40% (x=0.4) and a degree of grafting of DOTAGA and of DFO of 11% in All (y=0.11) Starting from a Chitosan with an Average Weight of 250 kDa

The chitosan-DOTAGA was synthesized beforehand as described in example 1.

A volume of 720 ml of purified chitosan-DOTAGA at a concentration of 7 g/l is placed in a 2 l round-bottomed flask. This solution (pH between 6 and 6.5) is made up to a total volume of 900 ml with ultrapure water. In parallel, 143.1 mg of p-NCS-Bz-DFO are weighed out and dissolved in 100 ml of DMSO. This solution is then added dropwise to the chitosan-DOTAGA solution. The solution is kept stirring and heated at 40° C. overnight. The solution is then purified with ultrapure water using the Sartoflow Smart purification machine with a PES membrane having a cutoff threshold of 100 kDa until a degree of purification of 545 is obtained. The solution at a concentration of 5.1 g/l is then lyophilized.

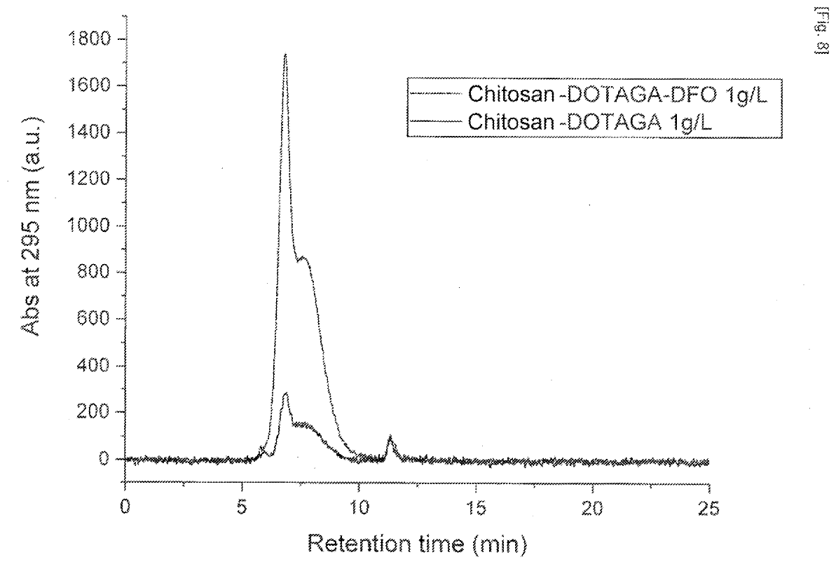

HPLC-UV analysis of the product makes it possible to confirm the grafting of the DFO and the elimination of the residual p-NCS-Bz-DFO. In fact, the comparison of the HPLC-UV analyses of the chitosan-DOTAGA and of the chitosan-DOTAGA-DFO shows an increase in the absorption of the polymer peak (around 7 min) when the p-NCS-Bz-DFO is grafted (FIG. 8).

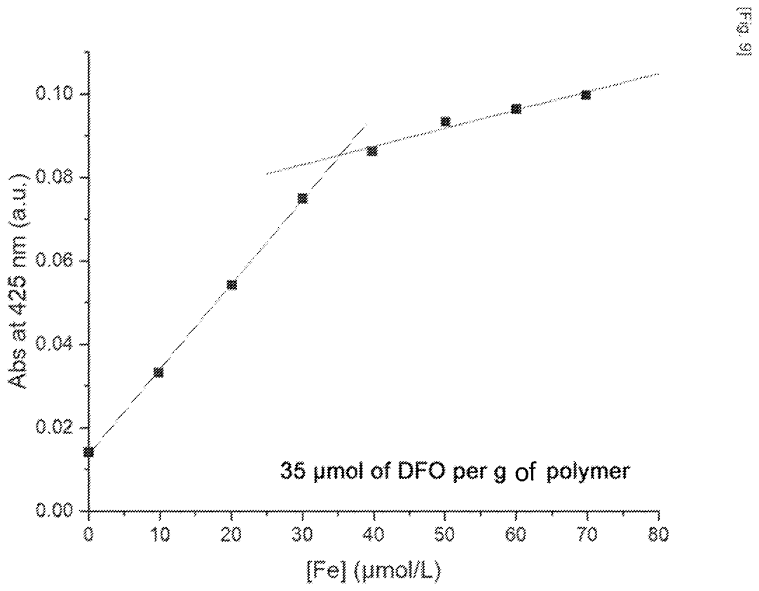

The amount of p-NCS-Bz-DFO grafted can be determined by UV-visible spectrophotometry quantitative analysis at the maximum absorption wavelength of the DFO-iron complex (425 nm). Increasing concentrations of iron(III) are added to a solution of chitosan-DOTAGA-DFO at 0.1 g/l in an acetate buffer at pH 4.5 (0.1M ammonium acetate and 0.1M acetic acid). The absorption measured at 425 mm is then plotted as a function of the iron concentration and a break in slope is observed for 35 μM of iron (FIG. 9). Since one molecule of DFO is able to complex a single iron atom, 1 gram of polymer thus contains 35 μmol of DFO. The chelating groups grafted thus contain 90% of DOTAGA and 10% of DFO (z=0.9).

1. A statistical polysaccharide with a weight-average molecular weight of between 100 kDa and 1000 kDa of formula I: 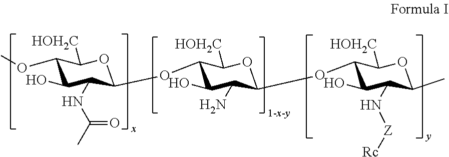
wherein
each Rc independently represents a group comprising a chelating agent, each Z independently represents a linker which is a single bond or a hydrocarbon-based chain comprising between 1 and 12 carbon atoms, wherein said hydrocarbon-based chain is optionally: linear or branched, comprises one or more unsaturations and to comprises one or more heteroatoms,
x is between 0.005 and 0.7,
y is between 0.01 and 0.7,
the y/x ratio being greater than or equal to 0.05, and
the sum x+y being greater than or equal to 0.30.

2. The polysaccharide as claimed in claim 1, characterized in that each Rc group is independently chosen from the group consisting of DOTA, NOTA, NODAGA, DOTAGA, DOTAM, NOTAM, DOTP, NOTP, TETA, TETAM, DTPA and DFO DOTAGA.

3. The polysaccharide as claimed, in claim 1, characterized in that it is of formula II 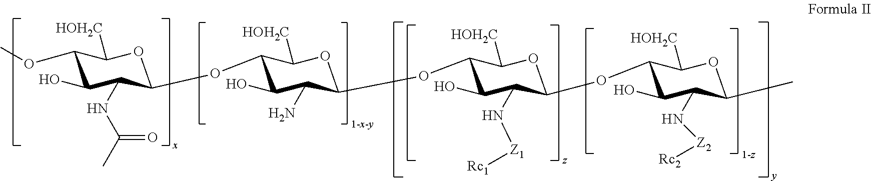
wherein:
Rc₁ and Rc₂ are different, and are groups comprising a chelating agent, Z₁ and Z₂, which may be identical or different, are linkers which may be a single bond or a hydrocarbon-based chain comprising between 1 and 12 carbon atoms, and wherein said hydrocarbon-based chain is optionally: linear or branched comprises one or more unsaturations and comprises one or more heteroatoms,
x is between 0.005 and 0.7,
y is between 0.01 and 0.7,
the y/x ratio being greater than or equal to 0.05,
the sum x+y being greater than or equal to 0.30, and
z is between 0.5 and 1.

4. The polysaccharide as claimed in claim 3, characterized in that Rc₁ and Rc₂ are independently chosen from the group consisting of DOTA, NOTA, NODAGA, DOTAGA, DOTAM, NOTAM, DOTP, NOTP, TETA, TETAM, DTPA and DFO.

5. The polysaccharide as claimed in claim 1, characterized in that x is between 0.25 and 0.4.

6. The polysaccharide as claimed in claim 1, characterized in that y is between 0.05 and 0.2.

7. The polysaccharide as claimed in claim 1, characterized in that it the polysaccharide is formulated in a composition in hydrogel form.

8. A method of capturing at least one metal, present in a subject comprising dialyzing a biological fluid of said subject with a dialysis fluid comprising the polysaccharide as claimed claim 1, wherein the polysaccharide chelates said at least one metal.

9. The polysaccharide as claimed in claim 1, characterized in that the polysaccharide chelates at least one metal.

10. An MRI imaging process comprising a step of administering the polysaccharide as claimed in claim 9.

11. A brachytherapy method comprising a step of administering a therapeutically effective amount, in a subject in need thereof, of the polysaccharide as claimed in claim 9, wherein said at least one metal is a radioactive isotope.

12. A method for preventing foodstuffs forgeries comprising a step of marking said foodstuffs with the polysaccharide as claimed in claim 9.

13. A process for obtaining the polysaccharide as claimed in claim 1 from chitosan, comprising at least the following three successive steps:
step 1: solubilization of a chitosan in an acid solution at a pH of between 4 and 5;
step 2: partial acetylation of the amine functions of the chitosan solubilized in step 1;
step 3: functionalization of at least one portion of the amine functions which were not acetylated in step 2.
